SEQUENCE LISTING (4 sequences)

SEQ ID NO: 1 - DNA, 22 bp, Homo sapiens
gtgcacctgactcctgaggaga

SEQ ID NO: 2 - DNA, 21 bp, Homo sapiens
ccttgataccaacctgcccag

SEQ ID NO: 3 - DNA, 23 bp, Homo sapiens
tcctgcttatccaaattcaccat

SEQ ID NO: 4 - DNA, 28 bp, Homo sapiens
acttccctctgacattacctgataattg

(19) United States
(12) Patent Application Publication
(10) Pub. No.: US 2011/0183338 A1
(43) Pub. Date: Jul. 28, 2011

(54) TWO STAGE ENRICHMENT OF CELL-FREE FETAL DNA IN MATERNAL PLASMA

(75) Inventor: Farideh Bischoff, Sugar Land, TX (US)

(73) Assignee: NOVARTIS VACCINES AND DIAGNOSTICS, INC., Emeryville, CA (US)

(21) Appl. No.: 12/865,381

(22) PCT Filed: Jan. 30, 2009
(86) PCT No.: PCT/US2009/032614

§ 371 Date: Feb. 22, 2011

Related U.S. Application Data
(60) Provisional application No. 61/024,872, filed on Jan. 30, 2008.

(51) Int. Cl. C12Q 1/68; C07H 21/00; C12P 19/34; C12N 9/16

(52) U.S. Cl. 435/6.12; 536/23.1; 435/91.2; 435/196; 435/6.1

(57) ABSTRACT

The present invention provides methods for enriching fetal nucleic acid in a biological sample from a maternal host. Also provided are methods for detecting the presence or absence of markers in fetal, tumor, or neoplastic nucleic acid. The methods can include treating the biological sample with DNase and, optionally, performing whole genome amplification on the treated samples. The biological sample can be, for example, a blood sample.

### RELATED APPLICATIONS

This application is a submission under 35 U.S.C. 371 of PCT/US2009/032614, filed Jan. 30, 2009 which claims priority to U.S. Provisional Application No. 61/024,872, filed Jan. 30, 2008, all of which are hereby incorporated by reference in their entirety.

Though fetal DNA is present in maternal plasma, the levels are relatively low to enable routine prenatal genetic analysis. Without being bound to any theory, we believe that a portion of fetal DNA is packaged and thus protected from plasma endo-nucleases. We propose a novel enrichment technique that combines two distinct methods. Separated plasma is first treated with DNase to effectively decrease the percentage of contaminating cell-free maternal DNA fragments. Subsequent increase of fetal sequences is achieved using a modified Whole Genome Amplification (WGA) technique.

Methods: Plasma was isolated from maternal whole blood (n=24) then treated with increasing concentrations of DNase. DNase treated DNA was further processed using the Qiagen DNA extraction mini kit prior to a modified WGA protocol to enrich smaller DNA fragments. Presence of fetal sequences was confirmed using real-time Taqman PCR (RT-PCR) to measure β-Globin and DYS1 sequence levels.

Results: Fetal DNA sequences were detected following all DNase treatments. Correct detection of male fetuses was achieved in all samples confirmed to have a male fetus (n=10) both before and after WGA. In addition to correct gender determination, the samples (n=7) that were subjected to the highest amount of DNase as well as the modified WGA protocol demonstrated significant enrichment of fetal sequences, attaining 50% fetal DNA.

Conclusions: Results confirm that fetal DNA in plasma is protected and resistant to degradation from DNase treatment. These preliminary data also suggest that an optimal level of DNase treatment can be achieved that allows further enrichment using WGA. The observation that DNase eliminates predominantly maternal sequences suggests that cell-free fetal DNA is packaged differently than the maternal counterpart, allowing preferential enrichment of fetal sequences.

### INTRODUCTION

Prenatal genetic diagnosis has relied on invasive procedures such as amniocentesis or chorionic villous sampling (CVS). While these procedures have provided reliable results for many years they still carry a slight risk to the fetus (1). Since the discovery of amplifiable fetal cell-free nucleic acids in maternal plasma (2), there have been numerous studies aimed at determining the potential for clinical non-invasive prenatal genetic tests. While many of these studies have shown great promise, the small existing quantities of fetal DNA has made it difficult to implement clinically. Therefore, we have focused on improving methods of fetal DNA isolation and enrichment. Without being bound to any theory, it is believed that these circulating nucleotides are the result of fetal cells undergoing apoptosis (3). The relative stability of cell-free DNA and RNA in plasma, which is known to contain nucleases, suggests that these nucleic acids are circulating within membrane bound vesicles formed as a result of the mechanism of programmed cell death (4). These fetal DNA fragments are also distinguishable from maternal fragments based on size, fetal fragments generally smaller (<300 bp) than maternal fragments (>500 bp) (5; Jorgez C et al., 2007).

We describe a novel two stage method for enrichment of fetal fragments. The first stage involves treatment of total maternal plasma (containing both maternal and fetal DNA fragments) with DNase. Given that fetal fragments are more stable and likely packaged by membrane bound apoptotic bodies, we hypothesize that DNase treatment would deplete the overall (unpackaged) maternally derived sequences. The second stage involves a modified whole genome amplification (WGA) protocol designed to amplify smaller fragments, presumably fetal.

### Methods

Following IRB approval and written informed consent, a total of 24 whole blood samples (10 confirmed male pregnancies, mean gestational age 18 1/7 weeks ranging from 11 4/7 to 25 2/7 weeks; 12 confirmed female pregnancies, mean gestational age 20 1/14 weeks ranging from 9 6/7 to 37 4/7 weeks; and 2 non-pregnant controls, 1 male and 1 female) were collected. For each, approximately 30 ml of blood drawn in ACD vacutainers was processed by an initial centrifugation at 800 g for 10 min to separate plasma from the cellular fraction. The plasma fraction was removed and centrifuged again at 16,000 g for 10 min to further remove any contaminating cellular particles. This fraction was then frozen in 800 μl aliquots and later thawed for simultaneous batch processing. Each 800 μl plasma sample was thawed at room temperature then subjected to DNase (Promega, Cat #M6101) treatment at various concentrations (untreated, 1 μL, 5 μL, 10 μL, 30 μL of 1 unit/μl). Samples were incubated at 37° C. for 1 hour before adding stop solution. Samples were next subjected to DNA extraction using the Qiagen QiAamp Blood Mini Kit (Cat #51106) and eluted in a final volume of 100 μL. With slight modifications, the protocol for the GenomePlex® Complete Whole Genome Amplification (WGA) Kit (Sigma, Cat #WGA2-50rxn) was followed on seven maternal blood samples (4 confirmed males, and 3 confirmed females). We modified the procedure by first omitting the manufacturer suggested fragmentation incubation due to the anticipated size of target sequences. Second the cycle number in the amplification step was increased to 20 rather than the suggested 14. Amplified samples were stored at 4° C. until RT-PCR analysis for detection and quantification of β-Globin (BGLO354F: GTG CAC CTG ACT CCT GAG GAG A, BGLO455R: CCT TGA TAC CAA CCT GCC CAG) (6) and DYS1 (DYS1F: TCC TGC TTA TCC AAA TTC ACC AT, DYS1R: ACT TCC CTC TGA CAT TAC CTG ATA ATT G) (7) (Applied Biosystems 7700, Foster City, Calif.). Level of enrichment was determined based on % fetal DNA which was calculated as a ratio of DYS1 to β-Globin. β-Globin represents the total amount of isolated DNA, maternal and fetal, while in the confirmed male pregnancies DYS1 represents the amount of fetal DNA present in the sample.

### Results

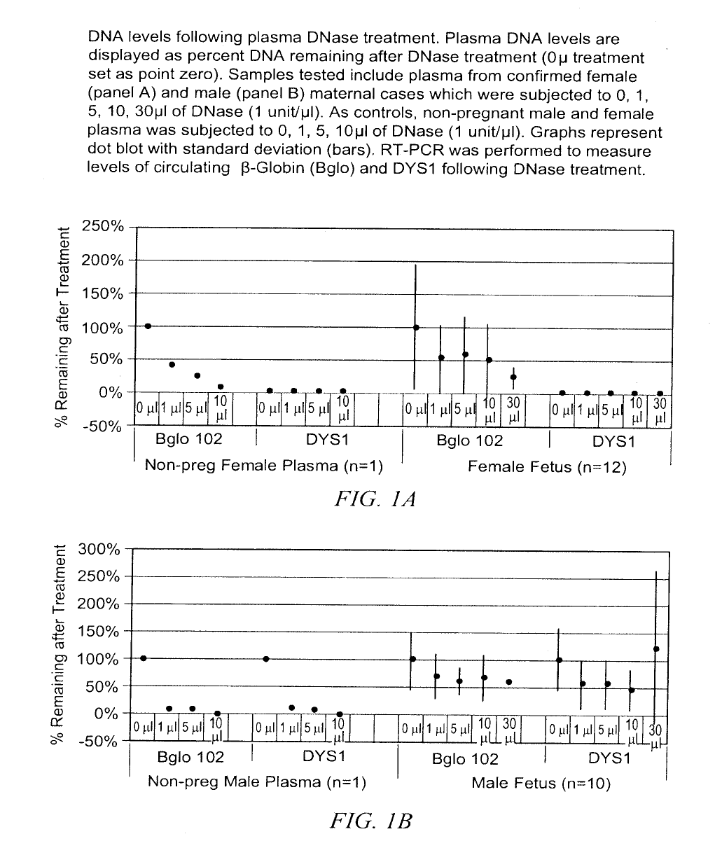

Following initial DNase treatments in control non-pregnant samples, both β-Globin and DYS1 levels decreased as a function of the amount of enzyme added. Each was effectively eliminated by DNase treatment. (FIG. 1). In maternal samples, although detected levels of β-Globin were decreased, these levels were persistent despite harsher DNase treatments (916.5±91.2 Geq/ml for male maternal cases and 610.5±389.62 Geq/ml for female maternal cases). No false positive DYS1 levels were detected in any of the known female pregnancies. However, among pregnancies with known male fetuses (n=10), there was 100% detection of DYS1 sequences at all treatment increments (0 μl: 127.4±72.3 Geq/ml, 1 μl: 71.4±57.3 Geq/ml, 5 μl: 71±58.6 Geq/ml, 10 μl: 57.1±46.6 Geq/ml, 30 μl: 154±179.6 Geq/ml).

WGA was performed on seven DNase treated maternal samples (3 female and 4 male). Though fetal sequences are detected in all samples, increased levels of fetal DYS1 sequences were only observed in the samples that were subjected to 30 μl of DNase (0 μl: 1979±2083.9 Geq/ml, 1 μl: 1.62±1.6 Geq/ml, 5 μl: 723.5±875.7 Geq/ml, 10 μl: 0.83±1.22 Geq/ml, 30 μl: 7025±4381 Geq/ml). Thus indicating that more stringent DNase treatments were necessary to diminish maternal sequences (based on β-Globin) to the extent that they were not present in levels that out-compete amplification of DYS1 sequences. In the samples that were subjected to 30 μl of DNase followed by the modified WGA protocol, we were able to achieve a mean value of 49.96% fetal DNA. In the samples that were not treated with DNase and only subjected to the modified WGA, the mean percent fetal DNA among samples was 11.16%. The samples that were treated with either 1, 5, 10 μl of 1 unit/μl of DNase all had mean values of 0.01%, 3.5%, and 0.01% fetal DNA respectively after WGA. Prior to WGA the samples had percentages ranging from 0.05% to 0.17%.

### DISCUSSION

Our results demonstrate that cell-free fetal DNA is resistant to degradation by DNase, supporting the hypothesis that cell-free fetal DNA is packaged in membrane bound vesicles. We effectively removed maternal sequences as well as any contaminating sequences that may lead to false-positive results. The levels of cell-free fetal DNA persist in patient samples versus control samples. This finding confirms that fetal sequences are resistant to degradation and protected or packaged differently than maternal sequences. β-Globin levels represent total DNA (maternal and fetal), thus failure to detect complete digestion of β-Globin is not surprising given that a portion is likely to be fetal. Fetal sequences appear to have a unique molecular characteristic, differentiating it from maternal sequences and enabling enrichment. Detection of all male cases and no false positives suggests that DNase treatment has a novel application in eliminating unwanted maternal sequences that have been degraded further while in circulation.

The GenomePlex® Complete Whole Genome Amplification Kit (Sigma-Aldrich) amplifies genomic DNA by first randomly fragmenting the DNA and then attaching a common sequence on the end which is used to amplify all the fragments by polymerase chain reaction (PCR). We eliminated the fragmentation step from the procedure to prevent larger maternal sequences from fragmenting for subsequent amplification. This potentially provides small pre-existing fragmented fetal sequences an advantage during amplification. WGA increased the fetal to maternal ratio in samples that underwent the most stringent DNase treatments. Prior to WGA, the mean % of fetal DNA was <1% at all treatment levels. However, 50% fetal DNA was achieved in samples that underwent 30 μl of DNase along with the modified WGA protocol, suggesting that this is a viable method of enrichment of cell-free fetal DNA in maternal plasma. Based on β-Globin levels, DNase appears to reduce the quantity of maternal sequences present in the sample, allowing fetal sequences to be amplified. The degradation of maternal nucleic acids prevents competition during amplification of fetal sequences in the PCR reaction. The samples that underwent milder (1, 5, 10 μl) DNase treatments displayed lower fetal to maternal ratios after WGA than samples that were not treated with DNAse. One possible explanation is that in these samples the larger maternal sequences were degraded but not completely eliminated, in effect replacing the fragmentation step of the original WGA protocol which allowed more efficient amplification of the maternal sequences.

We describe a combination of methods that allows us to overcome two major complications that has limited non-invasive prenatal DNA genetic testing: low fetal to maternal ratio and small quantity of fetal DNA. Overall, this novel two stage enrichment process shows great potential in selective enrichment followed by amplification.

### ACKNOWLEDGMENTS

NIH grant

### REFERENCES

1. Tabor A, Philip J, Madsen M, Banq J, Obel E B, Nørgaard Pedersen B. Randomised controlled trial of genetic amniocentesis in 4606 low-risk women. Lancet 1986; 8493:1287-93.
2. Lo Y M, Corbetta N, Chamberlain P F, Rai V, Sargent I L, Redman C W and Wainscoat J S. Presence of fetal DNA in maternal plasma and serum. Lancet 1997; 350:485-7.
3. Bischoff F Z, Lewis D E, Simpson J L. Cell-free fetal DNA in maternal blood: kinetics, source and structure. Human Reproductive Update 2004; 11:59-67.
4. Orozco A F, Bischoff F Z, Horne C, Popek E, Simpson J L, Lewis D E. Hypoxia-induced membrane-bound apoptotic DNA particles: potential mechanism of fetal DNA in maternal plasma. Ann N Y Acad Sci. 2006; 1075:57-62.
5. K. C. Allen Chan, Jun Zhang, Angela B. Y. Hui, Nathalie Wong, Tze K. Lau, Tse N. Leung, Kwok-Wai Lo, Dolly W. S. Huang, and Y. M. Dennis Lo. Size Distributions of Maternal and Fetal DNA in Maternal Plasma. Clinical Chemistry 2004; 500:88-92.
Carolina J. Jorgez, Farideh Z. Bischoff. Improving Utility of Circulating DNA for Prenatal Genetic Testing: Fragment Size and Purity. 2007
6. Y M Lo, M S Tein, T K Lau, C J Haines, T N Leung, P M Poon, J S Wainscoat, P J Johnson, A M Chang, and N M Hjelm. Quantitative analysis of fetal DNA in maternal plasma and serum: implications for noninvasive prenatal diagnosis. Am J Hum Genet. 1998; 62:768-75.
7. Tuangsit Wataganara, Erik LeShane, Antonio Farina, Geralyn M. Messerlian, Thomas Lee, Jacob A. Canick, Diana W. Bianchi. Maternal Serum Cell-Free DNA Levels are Increased in Cases of Trisomy 13 but not 18. Hum Genet 2003; 112:204-8

1. A method for enriching fetal nucleic acid comprising
treating a biological sample of a maternal host containing cell-free fetal nucleic acid with a composition comprising an agent with DNase activity,
wherein a first percentage of fetal nucleic acid in the biological sample prior to the treatment is lower than a second percentage of fetal nucleic acid in the biological sample after the treatment.

2. The method of claim 1, wherein the biological sample is a blood sample of a maternal host.

3. The method of claim 1, wherein the biological sample is a plasma or serum sample of a maternal host.

4. The method of claim 1, wherein the agent with DNase activity is DNase.

5. The method of claim 1, wherein the agent with DNase activity is DNase and the amount of DNase is from about 10 to 200 unit/μl

6. The method of claim 1, wherein the second percentage is from about 10% to 50%.

7. The method of claim 1, wherein the second percentage is at least 10%, 20%, 30%, 40%, or 50%.

8. The method of claim 1, wherein a first percentage of maternal nucleic acid in the biological sample prior to the treatment is higher than a second percentage of maternal nucleic acid in the biological sample after the treatment.

9. The method of claim 1 further comprising amplifying fetal nucleic acid in the biological sample after the treatment.

10. The method of claim 1 further comprising amplifying fetal nucleic acid in the biological sample after the treatment via a Whole Genome Amplification (WGA) method.

11. The method of claim 10, wherein the WGA method is conducted without fragmentation incubation.

12. A mixture obtained by the method of claim 1.

13. A mixture obtained by the method of claim 1, wherein the mixture contains at least 10%, 20%, 30%, 40%, or 50% fetal nucleic acid.

14. A method for detecting the presence or absence of a marker in fetal nucleic acid comprising
treating a biological sample of a maternal host containing cell-free fetal nucleic acid with a composition comprising an agent with DNase activity,
amplifying fetal nucleic acid in the biological sample after the treatment, and
detecting the presence or absence of a marker in fetal nucleic acid during or after amplification.

15. The method of claim 14, wherein the biological sample is a plasma or serum sample.

16. The method of claim 14, wherein the agent is DNase.

17. The method of claim 14, wherein the amplification is conducted via Whole Genome Amplification (WGA) method.

18. The method of claim 17, wherein the WGA method is conducted without fragmentation incubation.

19. The method of claim 14, wherein the percentage of fetal nucleic acid after the amplification is at least 10%, 20%, 30%, 40%, or 50%.

20. A method for enriching cell free nucleic acid from apoptotic or necrotic cells comprising
treating a biological sample containing cell free nucleic acid from apoptotic or necrotic cells with a composition comprising an agent with DNase activity,
wherein a first percentage of cell free nucleic acid from apoptotic or necrotic cells prior to the treatment is lower than a second percentage of cell free nucleic acid from apoptotic or necrotic cells in the biological sample after the treatment.

21. The method of claim 20, wherein the biological sample is a blood sample, plasma sample or serum sample.

22. The method of claim 20, wherein the agent with DNase activity is DNase.

23. The method of claim 20, wherein the agent with DNase activity is DNase and the amount of DNase is from about 10 to 200 unit/μl.

24. The method of claim 20, wherein the cell free nucleic acid is from tumor cells or neoplastic cells.

25. The method of claim 20 further comprising amplifying cell free nucleic acid in the biological sample after the treatment via a Whole Genome Amplification (WGA) method.

26. A method for detecting the presence or absence of a marker in a tumor or neoplastic nucleic acid comprising
treating a biological sample containing cell free nucleic acid from tumor or neoplastic cells with a composition comprising an agent with DNase activity,
amplifying cell free nucleic acid in the biological sample after the treatment, and
detecting the presence or absence of a marker in tumor or neoplastic nucleic acid during or after amplification.

27. The method of claim 26, wherein the biological sample is a blood sample, plasma sample or serum sample.

28. The method of claim 26, wherein the agent is DNase.

29. The method of claim 26, wherein the amplification is conducted via Whole Genome Amplification (WGA) method.

30. The method of claim 26, wherein the WGA method is conducted without fragmentation incubation.
